(19) United States
(12) Patent Application Publication
(10) Pub. No.: US 2005/0042210 A1
(43) Pub. Date: Feb. 24, 2005

(54) Body tissue filling material, production method thereof and body tissue filler

(75) Inventor: Hidemi Akai, Tokyo (JP)

Correspondence Address: SCULLY SCOTT MURPHY & PRESSER, PC, 400 GARDEN CITY PLAZA, GARDEN CITY, NY, 11530, US

(73) Assignees: Hidemi Akai, Tokyo (JP); OLYMPUS CORPORATION, Tokyo (JP)

(21) Appl. No.: 10/881,488

(22) Filed: Jun. 30, 2004

(30) Foreign Application Priority Data
Jul. 2, 2003 (JP) 2003-270526

(51) Int. Cl. A61K 45/00; A61K 33/42

(52) U.S. Cl. 424/93.72; 424/602

(57) ABSTRACT

In this body tissue filling material, platelet-rich plasma is mixed into granular β-tricalcium phosphate. The platelet-rich plasma is produced by a method consisting of a step in which blood is housed in a first vessel, the inside of which has been sterilized, a step in which blood cells are separated from the blood by allowing centrifugal force to act on the first vessel in which the blood is housed, a step in which the liquid remaining after separating the blood cells is transferred to a second vessel aseptically connected to the first vessel, a step in which the connection between the first vessel and the second vessel is sealed, a step in which plasma is separated from this liquid by allowing centrifugal force to act on the second vessel that houses the liquid, and a step in which the remaining platelet-rich plasma from which plasma has been separated is transferred to a third vessel aseptically connected to the second vessel.

### BACKGROUND OF THE INVENTION

1. Field of the Invention

The present invention relates to a body tissue filling material, its production method and a body tissue filler, and more particularly, to a preferable technology used to reconstruct a cleft jaw in cleft palate.

The present application claims priority from Japanese Patent Application No. 2003-270526 filed on Jul. 2, 2003, and cites the contents of that publication herein.

### DESCRIPTION OF THE RELATED ART

Treatment of cleft palate consists of performing a bone graft for the purpose of reconstructing the cleft portion of the jaw. This bone graft began with primary or early bone grafts, followed by late bone grafts for the purpose of retention in the 1960s, and finally progressed to secondary cleft jaw bone grafts for the purpose of guiding the cuspids in the 1970s, with this type of bone graft still being employed at present.

More recently, reports have also been observed describing simultaneous reconstruction of cleft jaw during single-stage surgery for cleft palate (see, for example, Platelet-Rich Plasma (PRP), [Search date: May 9, 2003], Internet web site <URL:http://home.att.nejp/iota/dental/newpage29.htm>). Examples of graft materials include autoplastic bone such as the rib, skull, tibia, mandible and ilium, and heteroplastic bone such as freeze-dried homoplastic stored bone. In addition, hydroxyapatite (HAP) is used as artificial bone.

In recent years, platelet-rich plasma (to be referred to as PRP) has come to be known to contain various autologous growth factors such as TGF-β1, PDGF and IGF-1, and wound healing has been reported to be accelerated during bone and soft tissue grafts using PRP. More recently, the importance of PRP is increasing due to advancements made in the fields of regenerative medicine and tissue engineering and their resulting clinical applications.

### SUMMARY OF THE INVENTION

The body tissue filling material including: granular β-tricalcium phosphate, and platelet-rich plasma mixed into the β-tricalcium phosphate.

The body tissue filling material of the present invention preferably contains the platelet-rich plasma at 10-60% by weight.

In the body tissue filling material of the present invention, the particle diameter of the β-tricalcium phosphate is preferably 0.1-10 mm.

The production method of the body tissue filling material of the present invention is included of a step in which blood cell components are separated by allowing centrifugal force to act on blood housed in a first vessel, the inside of which has been sterilized, a step in which the liquid remaining after separating the blood cell components is transferred to a second vessel aseptically connected to the first vessel, a step in which, after the remaining liquid has been transferred to the second vessel, the plasma component is separated by allowing centrifugal force to act on the remaining liquid to extract platelet-rich plasma, and a step in which the extracted platelet-rich plasma is mixed into granular β-tricalcium phosphate.

In the production method of the body tissue filling material of the present invention, centrifugal force is preferably allowed to act for an amount of time of 15-25 minutes at a rotating speed of 1000-2500 rpm in the step in which blood cell components are separated.

In the production method of the body tissue filling material of the present invention, the plasma component is preferably separated by allowing centrifugal force to act for an amount of time of 10-20 minutes at a rotating speed of 1500-3500 rpm.

The body tissue filler of the present invention is included by mixing bone marrow cells into a body tissue filling material in which platelet-rich plasma is mixed into granular β-tricalcium phosphate.

The body tissue filler of the present invention is included by mixing mesenchymal stem cells into a body tissue filling material in which platelet-rich plasma is mixed into granular β-tricalcium phosphate.

The body tissue filler of the present invention is preferably mixed with a biocompatible adhesive. In addition, the adhesive is preferably fibrin glue.

### BRIEF DESCRIPTION OF THE DRAWINGS

FIG. 1 is a front view showing a PRP extraction vessel used in the production of a body tissue filler as claimed in a first embodiment of the present invention.

FIG. 2 is a flow chart showing a production method for producing PRP using the extraction vessel shown in FIG. 1.

FIGS. 3A through 3F are schematic drawings showing each of the work steps according to the flow chart of FIG. 2.

FIG. 4 is a schematic drawing showing the state in which the body tissue filler as claimed in a first embodiment of the present invention is filled into a cleft jaw.

FIG. 5 is an enlarged view for explaining the restorative action of the cleft jaw in FIG. 4.

FIG. 6 is a CT tomograph showing the state of restoration of a cleft jaw according to an example of the present invention.

FIG. 7 is a CT tomograph showing the state of restoration of a cleft jaw according to a comparative example of the present invention.

### DETAILED DESCRIPTION OF THE PREFERRED EMBODIMENTS

The following provides an explanation of the body tissue filling material, its production method and the body tissue filler as claimed in a first embodiment of the present invention.

The body tissue filling material as claimed in the present embodiment is a bone filling material and consists of a mixture of β-tricalcium phosphate and PRP.

Granular β-tricalcium phosphate having a particle diameter of 0.1-10 mm is used for the β-tricalcium phosphate. Each granule of β-tricalcium phosphate is provided with a large number of fine pores of roughly 100-400 μm, and is in the form of a porous body having porosity of 75% or more.

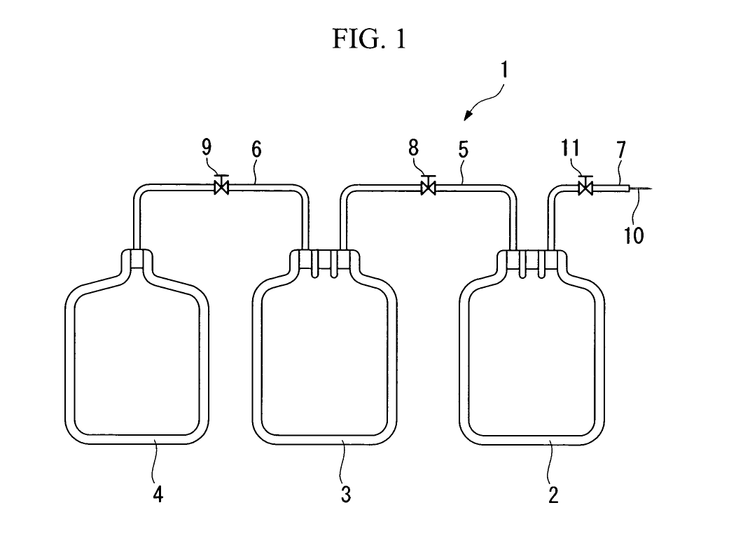

The PRP is extracted according to the following method. Namely, as shown in FIG. 1, blood is housed in a first vessel, the inside of which has been sterilized (S1), blood cells are separated from the blood by allowing centrifugal force to act on the first vessel housing the blood (S2), the liquid remaining after separating the blood cells is transferred to a second vessel aseptically connected to the first vessel (S3), the connection between the first vessel and the second vessel is sealed (S4), plasma is separated from the liquid by allowing centrifugal force to act on the second vessel housing this liquid (S5), and the plasma is separated into a third vessel aseptically connected to the second vessel and the remainder-is used as platelet-rich plasma (S6).

The PRP is specifically extracted according to the following method. As shown in FIG. 1, extraction vessel 1 is prepared provided with three vessels 2, 3 and 4, two tubes 5 and 6 connected to these vessels 2, 3 and 4, and a blood collection tube 7 connected to one vessel 2.

Vessels 2, 3 and 4 and tubes 5, 6 and 7 are integrally formed from, for example, vinyl chloride. Vessels 2, 3 and 4 are composed by comparatively thin, flexible sheets, and the periphery is sealed by heat fusion. As a result, vessels 2, 3 and 4 can be easily deformed by pressure from the outside.

Tubes 5 and 6 are respectively connected to vessel 2, 3 or 4 so that both ends open into vessels 2, 3 and 4. In addition, valves 8 and 9 are respectively provided at intermediate locations of tubes 5 and 6 so as to close or open these tubes 5 and 6.

In addition, blood collection tube 7 is connected to vessel 2 so that one end opens into vessel 2, and is provided with a syringe needle 10 on the other end.

The insides of vessels 2, 3 and 4 and tubes 5, 6 and 7 as well as syringe needle 10 are sterilized.

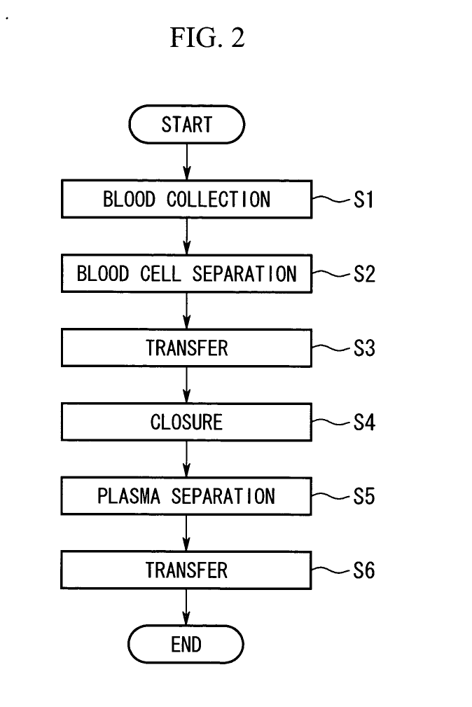

In order to produce the PRP as claimed in the present embodiment, as shown in FIG. 2, a first step S1, in which syringe needle 10 is inserted into a patient to collect blood in vessel 2, a second step S2 in which blood cells are separated from the blood by allowing centrifugal force to act on vessel 2 in which the blood is housed, a third step S3 in which the remaining liquid after separating the blood cells is transferred to vessel 3, a fourth step S4 in which valve 8 between vessels 2 and 3 is closed, a fifth step S5 in which plasma is separated from the remaining liquid by allowing centrifugal force to act on vessel 3, and a sixth step S6 in which the remaining platelet-rich plasma separated from the plasma is transferred to vessel 4, are carried out.

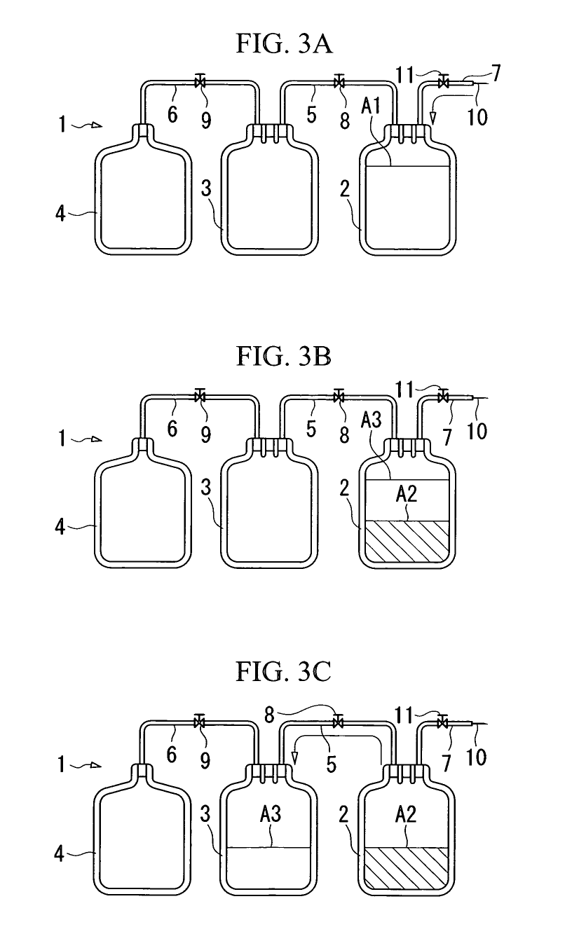

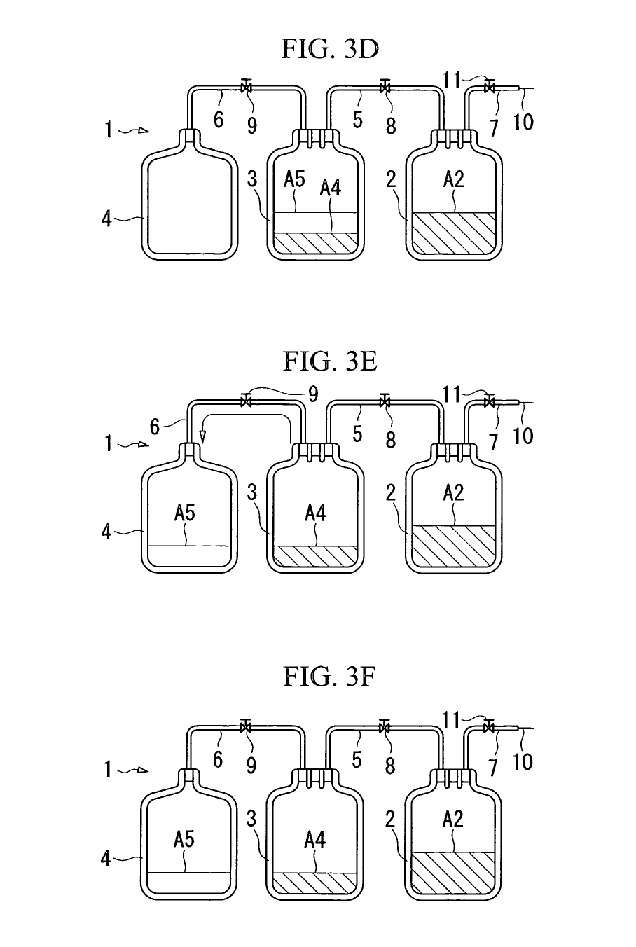

In the first step S1, as shown in FIG. 3A, negative pressure is generated within vessel 2 so that blood A1 flows into vessel 2. Negative pressure should be generated by, for example, aspirating the outer surface of vessel 2 from the outside so as to expand the inner volume. At this time, a predetermined amount of citric acid anticoagulant and so forth should be injected into vessel 2 so that the collected blood A1 does not coagulate.

As shown in FIG. 3B, the second step S2 is carried out by placing vessel 2 in a centrifuge (not shown) with blood A1 housed inside. In the centrifuge, vessel 2 is rotated for, for example, 20 minutes at a rotating speed of 1200 rpm. As a result, blood cells A2 present in blood A1 that are comparatively heavy are separated to the outside in the centrifugal direction. Thus, by arranging the side of vessel 2 at which tube 5 is connected to the inside in the centrifugal direction, the remaining liquid A3 after separating blood cells A2 from blood A1 (plasma+platelet-rich plasma) can be arranged on the side of tube 5.

As shown in FIG. 3C, the third step S3 is carried out by pressing on vessel 2 from the outside of vessel 2. Namely, since vessel 2 is made of a flexible sheet-like material and can be easily deformed by pressure from the outside, the contents of vessel 2 can be pushed out by easily deforming vessel 2 when pressed from the outside. Within vessel 2, since blood cells A2 are separated from the remaining liquid A3 by a well-defined separation plane P1 due to the centrifugation in the second step S2, by pressing vessel 2, the remaining liquid A3 other than blood cells A2 can be transferred from the inside of vessel 2 to vessel 3 through tube 5.

As shown in FIG. 3D, in the fourth step S4, tube 5 is closed by valve 8. As a result, liquid A3 that has been transferred to vessel 3 is prevented from flowing back into vessel 2.

As shown in FIG. 3E, the fifth step S5 is carried out by placing vessel 3 in a centrifuge with liquid A3 housed inside. In the centrifuge, vessel 3 is rotated for, for example, 15 minutes at a rotating speed of 1900 rpm. As a result, plasma A5 present in liquid A3 that is comparatively heavy is separated to the outside in the centrifugal direction. Thus, by arranging the side of vessel 3 at which tube 5 is connected to the inside in the centrifugal direction, platelet-rich plasma A4 can be arranged on the side of tube 5 in the form of the liquid remaining after separating plasma A5 from liquid A3. In addition, even in the case blood cells A2 are mixed with liquid A3 when liquid A3 is transferred from vessel 2 to vessel 3 in step S3, since blood cells A2 are heavier than plasma A5, they are separated without contaminating platelet-rich plasma A4.

As shown in FIG. 3F, the sixth step S6 is carried out by pressing vessel 3 from the outside of vessel 3. Thus, similar to the third step S3, platelet-rich plasma A4 is easily transferred from vessel 3 to vessel 4 through tube 5. As a result, platelet-rich plasma A4 is aseptically extracted.

A body tissue filling material as claimed in the present embodiment is therefore then produced by mixing granular β-tricalcium phosphate with the platelet-rich plasma A4 extracted in this manner.

According to the body tissue filling material as claimed in the present embodiment, when this body tissue filling material is filled into an area where body tissue is missing, growth of the body tissue contacted by the body tissue filling material is promoted due to the action of the platelet-rich plasma A4 contained therein. Namely, the body tissue grows using platelet-rich plasma A4 as a type of growth factor and β-tricalcium phosphate as a footing, thereby resulting in restoration of the area where body tissue is missing.

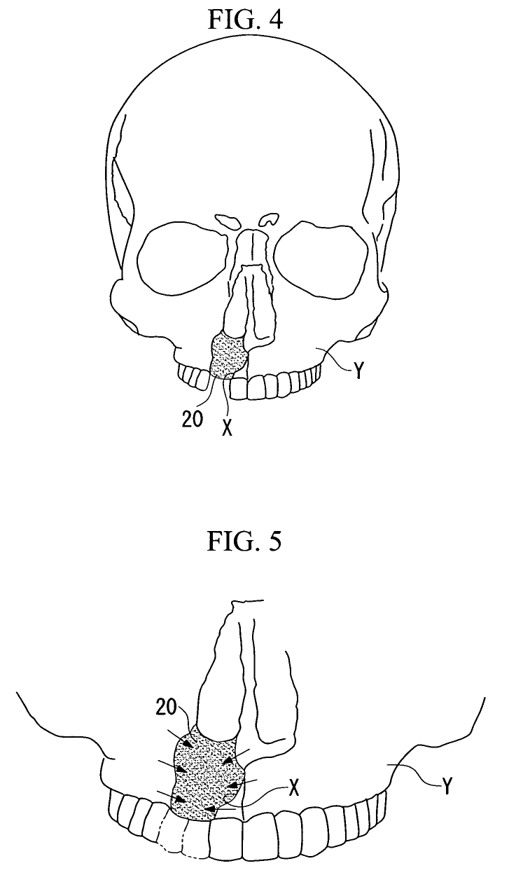

In addition, a body tissue filler is produced by mixing bone marrow cells into a body tissue filling material produced in this manner. According to this body tissue filler, the bone marrow cells can be grown from within the body tissue filler thereby enabling more efficient restoration of areas where body tissue is missing. Namely, as shown in FIG. 4, the body tissue filler is filled into a cleft jaw. In the body tissue filler filled into the cleft jaw, in addition to osteogenic action in the direction of the arrows in FIG. 5 from the two directions of the lateral surfaces in contact with the mandible, the bone marrow cells mixed inside also growth due to the action of PRP, thereby resulting in osteogenic action.

As a result, the cleft jaw can be restored more quickly as compared with the case of filling with β-tricalcium phosphate alone.

In addition, by composing the body tissue filler by mixing with a biocompatible adhesive such as fibrin glue, the β-tricalcium phosphate granules, bone marrow cells and PRP can be integrated into a mass, thereby making it possible to facilitate the task of filling the body tissue filler into the area where body tissue is missing.

Furthermore, although bone marrow cells are mixed into the body tissue filler as claimed in the present embodiment, mesenchymal stem cells may be mixed instead.

### EXAMPLE 1

The following provides an explanation of the case of using the body tissue filler as claimed in the present embodiment for the treatment of cleft palate.

A body tissue filler was produced by mixing the following components at the mixing ratios shown: β-tricalcium phosphate granules: 20 wt %, PRP: 40 wt %, bone marrow cells: 20 wt %, fibrin glue: 20 wt %.

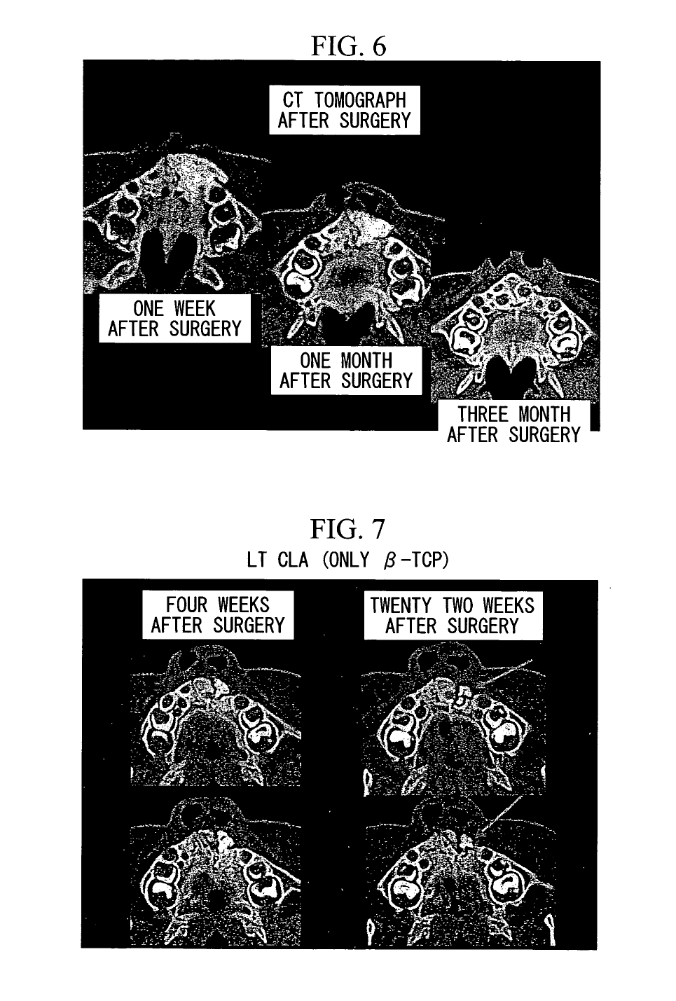

Status at one week, one month and three months after surgery is shown in FIG. 6 after filling the body tissue filler produced in this manner into the cleft jaw portion of a cleft palate. FIG. 6 is a CT tomograph obtained by CT of the cleft jaw. It can be seen from this tomograph that osteogenic action was occurring over a wide range at one month after surgery. In addition, it can also be seen that the cleft jaw was completely restored at three months after surgery, and that teeth were forming in the newly restored upper mandible.

For the sake of comparison, the case of arranging only β-tricalcium phosphate in the area where body tissue is missing is shown in FIG. 7. There is no occurrence of adequate osteogenic action even after four weeks have elapsed following surgery, and not only is there no formation of teeth, the cleft jaw can be seen to have not been adequately restored even at twenty two weeks after surgery.

According to the present invention, when the body tissue filling material is filled into an area where bone is missing, growth of bone tissue in contact with the body tissue filling material is promoted due to the action of platelet-rich plasma contained within the body tissue filling material. Since bone formation action is promoted by using granular β-tricalcium phosphate as a footing, even if applied to an area where body tissue is missing which makes little contact with body tissue as in the treatment of a cleft jaw, the area where body tissue is missing can be restored.

According to the present invention, as a result of separating blood in a first vessel and a second vessel that are isolated from the outside, pure platelet-rich plasma that is free of contamination by dust particles and so forth from the outside can be easily extracted, and as a result of mixing the extracted platelet-rich plasma into granular β-tricalcium phosphate, a body tissue filling material can be produced easily.

According to the present invention, a body tissue filling material can be produced by efficiently extracting platelet-rich plasma.

Although the above has provided an explanation of a preferred embodiment of the present invention, the present invention is not limited to this embodiment. Additions, omissions, substitutions and other alterations may be made to the present invention provided they are within a range that does not deviate from the gist of the present invention. The present invention is not limited by the aforementioned explanation, but is only limited by the attached scope of claim for patent.

1. A body tissue filling material comprising:
granular β-tricalcium phosphate, and
platelet-rich plasma mixed into the β-tricalcium phosphate.

2. A body tissue filling material according to claim 1 wherein, the platelet-rich plasma is contained at 10-60% by weight.

3. A body tissue filling material according to claim 1 wherein, the particle diameter of the β-tricalcium phosphate is 0.1-10 mm.

4. A production method of a body tissue filling material comprising:
a step in which blood cell components are separated by allowing centrifugal force to act on blood housed in a first vessel, the inside of which has been sterilized,
a step in which the liquid remaining after separating the blood cell components is transferred to a second vessel aseptically connected to the first vessel,
a step in which, after the remaining liquid has been transferred to the second vessel, the plasma component is separated by allowing centrifugal force to act on the remaining liquid to extract platelet-rich plasma, and
a step in which the extracted platelet-rich plasma is mixed into granular β-tricalcium phosphate.

5. A production method of a body tissue filling material according to claim 4 wherein, centrifugal force is allowed to act for an amount of time of 15-25 minutes at a rotating speed of 1000-2500 rpm in the step in which blood cell components are separated.

6. A production method of a body tissue filling material according to claim 4 wherein, centrifugal force is allowed to act for an amount of time of 10-20 minutes at a rotating speed of 1500-3500 rpm in the step in which the plasma component is separated to extract platelet-rich plasma.

7. A body tissue filler comprised by mixing bone marrow cells into a body tissue filling material according to claim 1.

8. A body tissue filler according to claim 7 wherein, a biocompatible adhesive is mixed.

9. A body tissue filler according to claim 9 wherein, the adhesive is fibrin glue.

10. A body tissue filler comprising by mixing mesenchymal stem cells into a body tissue filling material according to claim 1.

11. A body tissue filler according to claim 10 wherein, a biocompatible adhesive is mixed.

12. A body tissue filler according to claim 11 wherein, the adhesive is fibrin glue.
